(19) United States
(12) Patent Application Publication
(10) Pub. No.: US 2022/0151906 A1
(43) Pub. Date: May 19, 2022

(54) NON-THERAPEUTIC COSMETIC USE OF CARBOXYALKYLATED STARCH IN AN EMULSION FOR CUSHION AND SOFT FEEL SENSORY EFFECTS

(71) Applicant: ROQUETTE FRERES, Lestrem (FR)

(72) Inventors: Géraldine LOUVET-POMMIER, Lachelle (FR); Camille LACORE, Montmorency (FR); Sophie PIOT, Paris (FR)

(21) Appl. No.: 17/593,714

(22) PCT Filed: Mar. 30, 2020
(86) PCT No.: PCT/EP2020/058930

§ 371 Date: Sep. 23, 2021

(30) Foreign Application Priority Data
Mar. 29, 2019 (FR) FR1903357

(51) Int. Cl. A61K 8/73; A61K 8/06; A61K 8/34; A61K 8/36; A61K 8/37; A61K 8/31; A61K 8/92; A61Q 19/00

(52) U.S. Cl. CPC A61K 8/732; A61K 8/062; A61K 8/064; A61K 8/345; A61K 8/361; A61K 2800/591; A61K 8/31; A61K 8/342; A61K 8/922; A61Q 19/00; A61K 2800/436; A61K 8/37

(57) ABSTRACT

The invention relates to the field of cosmetics, in particular oil-in-water emulsions or water-in-oil emulsions, comprising a sensory agent having a “cushion” and a softness effect. The invention relates to the non-therapeutic cosmetic use of a carboxyalkylated granular starch in an oil-in-water or a water-in-oil emulsion as a sensory agent having a “cushion” effect and a softness effect. A cream prepared according to this use has a whipped cream texture.

### TECHNICAL FIELD

The invention relates to the field of cosmetics, in particular oil-in-water emulsions or water-in-oil emulsions, comprising a sensory agent having “cushion” effects and a “softness” effect.

### TECHNICAL PROBLEM

A current need among cosmetic product formulators is to have functional ingredients, in particular with sensory functionality, that are natural or of natural origin.

Users of cosmetic products are currently seeking a sensory experience referred to as “cushion effect”.

In a surprising and unexpected manner, the invention makes it possible to propose a cosmetic product with “cushion effect” having a sensory agent of natural origin.

### DESCRIPTION OF EMBODIMENTS

The invention relates to the non-therapeutic cosmetic use of a carboxyalkylated granular starch in an oil-in-water or a water-in-oil emulsion, preferentially oil-in-water, as a sensory agent having a cushion effect and a softness effect.

In other words, the invention relates to the use of a carboxyalkylated granular starch in an oil-in-water or water-in-oil emulsion, preferentially oil-in-water, so that said emulsion has a sensory profile characterized by a cushion effect, and a softness.

According to the invention, the cushion effect has a score greater than or equal to 7, preferentially greater than or equal to 7.5, the score being measured by evaluation by a sensory panel using the “EBltouch®” kit.

According to the invention, the softness effect has a score greater than or equal to 7, preferentially greater than or equal to 7.5, the score being measured by a sensory panel using the “EBltouch®” kit.

Carboxyalkylated Ganular Sarch

According to a first embodiment, the starch useful for the invention is a carboxyalkylated granular starch.

The starch may be a starch from corn, potato, wheat, rice, peas, oats, lentils, faba beans, broad beans, beans, chickpeas, cassava, or combinations thereof. Preferably, the starch is a potato starch.

By “granular” the applicant means that the starch is in the form of granules having a structure organized as alternations of crystalline, semi-crystalline and amorphous layers. This organization is generally characterized by the observation of birefringence patterns under exposure to polarized light, also known as “Maltese crosses”. It may also be characterized by a measurement of X-ray diffraction.

By “carboxyalkylated” the applicant means a starch that has been chemically modified by at least one carboxyalkylation.

“Carboxyalkylation” within the meaning of the present invention is understood, in particular and without this list being exhaustive, to refer to any chemical modification able to replace all or part of the O—H hydroxyl groups of the starch with groups of the O—(CH2)n-COOH type, of the O—(CH2)n-COOX type, of the O—CH-COOH-(CH2)m-COOH type, or of the O—CH-COOH-(CH2)m-COOX type, wherein:
n is equal to zero (cyanidation reaction) or greater, preferably comprised between 1 (carboxymethylation reaction) and 4 (carboxybutylation reaction) and most preferentially equal to 1 (carboxymethylation) or 2 (carboxyethylation reaction or cyanoethylation reaction)
m is greater than zero and in particular equal to 1 (carboxyalkylation using chloromalonic acid for example),
X is a cation, preferably monovalent, selected particularly from the group comprising sodium, potassium and lithium, in particular sodium and potassium, sodium being generally preferred.

Preferentially, the carboxyalkylated granular starch is selected from the carboxymethylated, carboxyethylated, carboxypropylated starches, or a mixture thereof. The carboxyalkylated granular starch useful for the invention may also have been modified by at least two chemical carboxyalkylation modifications, preferentially selected from carboxymethylation, carboxyethylation, carboxypropylation.

In a particularly advantageous fashion, the carboxyalkylation consists of a carboxymethylation reaction using monochloroacetic acid or one of the salts thereof, in particular sodium monochloroacetate.

Examples of carboxymethylated starch useful for the invention are Glycolys® or Explosol® marketed by Roquette Freres, Vivastar® or Explotab® by JRS Pharma.

Carboxyalkylated and Cross-Linked Granular Starch

According to a second embodiment, the starch useful for the invention is a carboxyalkylated and cross-linked granular starch. The carboxylkylated granular starch may be cross-linked by a cross-linking agent selected from polyfunctional acids, polyacid anhydrides, or polyfunctional basic organic molecules, as well as the metal salts thereof.

The polyfunctional acids that have cross-linked the carboxyalkylated starch may be polycarboxylic acids, such as citric acid, or polyphosphoric acids, such as triphosphoric acid; Polyacid anhydrides that have cross-linked the carboxyalkylated starch may be mixed polyacid anhydrides such as mixed adipic/acetic anhydride. The polyfunctional basic organic molecules that have cross-linked the carboxyalkylated starch may be polyphosphates. The metal salts thereof may be salts of sodium, manganese, calcium, manganese, iron, copper, zinc. A preferred alternative cross-linking agent is sodium salts of polyacids, such as sodium trimetaphosphate, sodium tripolyphosphate. Other cross-linking agents that have cross-linked the carboxyalkylated starch may be polyfunctional aldehydes, such as glyoxal; halogenated epoxides, such as epichlorohydrin; aliphatic or aromatic diisocyanates with an alkyl chain of less than 8 carbon atoms, such as hexamethylene diisocyanate; oxohalides, such as phosphorus oxychloride.

The carboxyalkylated granular and/or cross-linked starch is present in the emulsion at a percentage of 1% to 20%, preferentially of 3% to 18%, most preferentially of 5% to 15%, by weight relative to the total weight of said emulsion.

Emulsion

The emulsion according to the invention may be an oil-in-water emulsion obtained by emulsifying a fatty phase in an aqueous phase in the presence of surfactants, or a water-in-oil emulsion obtained by emulsifying an aqueous phase in a fatty phase in the presence of surfactants. Preferentially, the emulsion is an oil-in-water emulsion.

Fatty Phase

The fatty phase may contain at least one oil. For the purposes of the present invention, “oil” is understood to mean a compound which is liquid at room temperature (25° C.) and which, when it is introduced at an amount of at least 1% by weight into the water at 25° C., is not at all soluble in the water, or soluble to an extent of less than 10% by weight, relative to the weight of oil introduced into the water. The fatty phase may represent a weight percentage of 10% to 70%, by weight relative to the total weight of said emulsion.

The liquid fatty phase advantageously comprises one or more nonvolatile oils which afford an emollient effect on the skin. Mention may be made of fatty esters such as cetearyl isononoate, isotridecyl isononoate, isostearyl isostearate, isopropyl isostearate, isopropyl myristate, isopropyl palmitate, butyl stearate, hexyl laurate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, tracetin, tricaprin, caprylic/capric acid triglycerides, triheptanoin, coco caprate and caprylate mixture, benzoates of C12 to C15 alcohols, glycol esters such as butylene glycol cocoate, glyceryl triisostearate, tocopherol acetate, higher fatty acids such as myristic acid, palmitic acid, stearic acid, behenic acid, oleic acid, linoleic acid, linolenic acid or isostearic acid, higher fatty alcohols such as oleic alcohol, or a polymer such as diheptyl succinate/capryloyl glycerin/Sebacic acid copolymer, or vegetable oils such as avocado oil, camellia oil, hazelnut oil, tsubaki oil, cashew nut oil, argan oil, soybean oil, grape seed oil, sesame oil, corn oil, wheatgerm oil, rapeseed oil, sunflower oil, cottonseed oil, jojoba oil, peanut oil, olive oil and mixtures thereof, vegetable butters such as shea butter, camellia butter.

These oils may also be hydrocarbon type oils, for example C15-C19 alkanes, or silicone type oils such as paraffin oil, squalane, petroleum jelly, dimethyl siloxanes and mixtures thereof.

Preferably, the non-volatile oil is selected from coco-caprylate/caprate, triheptanoin, diheptyl succinate/capryloyl glycerin/Sebacic acid copolymer, C15-C19 alkanes, or the mixtures thereof.

The at least one non-volatile oil may be present in the emulsion with a percentage of 10% to 70%, by weight relative to the total weight of said emulsion.

The liquid fatty phase may also comprise volatile oils. Volatile oil is intended to mean an oil which is capable of evaporating from the skin in less than one hour at room temperature and atmospheric pressure. Volatile oils may for example be selected from silicone oils or C2-C6 short-chain fatty acid triglycerides in order to reduce the greasy feel. Volatile oils may be present in the emulsion with a percentage of 10% to 70%, by weight relative to the total weight of said emulsion.

The fatty phase of the emulsion according to the invention may further advantageously comprise at least one fatty phase structuring agent such as a lipophilic gelling agent, a wax and/or a pasty compound. The at least one fatty phase structuring agent may be present in the emulsion with a percentage of 1% to 20%, by weight relative to the total weight of said emulsion.

“Wax” is intended to mean a fatty substance having reversible liquid-solid state change and having a melting point greater than 30° C. and generally less than 90° C., which is liquid under the preparation conditions of the emulsion and which has anisotropic crystalline organization in the solid state. The waxes used according to the invention may consist of polar or apolar waxes or a mixture of these two. “Apolar” is intended to mean a wax containing only carbon, hydrogen and/or phosphorus atoms, and in particular a hydrocarbon.

The polar waxes may be selected from animal waxes, vegetable waxes, and synthetic or silicone waxes containing polar groups such as esters. Mention may thus be made of carnauba wax, candelilla wax, beeswax (Cera alba), Chinese insect wax (Ericerus pela), Japan wax, sumac wax, montan wax, triesters of C8-C20 acids and glycerin, such as glyceryl tribehenate, acetylated glycol stearate, sold particularly by VEVY under the trade name CETACENE, and mixtures thereof. These waxes may in particular be used in predispersed form in an oil, as is the case of the mixture of candelilla wax and jojoba seed oil. The waxes may be present in the emulsion with a percentage of 1% to 20%, by weight relative to the total weight of said emulsion.

“Pasty compound” is understood to mean lipophilic fatty substances which, like waxes, are capable of undergoing a reversible liquid-solid state change and which have, in the solid state, anisotropic crystalline organization, but which differ from waxes in that they contain, at a temperature of 23° C., a liquid fraction and a solid fraction. The pasty compounds may be present in the emulsion with a percentage of 1% to 20%, by weight relative to the total weight of said emulsion.

Aqueous Phase

The emulsion according to the invention may comprise an aqueous phase comprising water and optionally, one or more water-miscible organic solvents and/or one or more rheology agents. The aqueous phase may represent a weight percentage of 30 to 90% by weight relative to the total weight of said emulsion. When it is an oil-in-water emulsion, the weight percentage of aqueous phase preferentially ranges from 30 to 85%, more preferentially from 50 to 85%, by weight relative to the total weight of said emulsion. When it is a water-in-oil emulsion, the weight percentage of aqueous phase preferentially ranges from 30 to 70%, by weight relative to the total weight of said emulsion. Water may represent 30 to 90% by weight relative to the total weight of said emulsion. Preferably, the water is demineralized water.

The one or more water-miscible organic solvents may be present in the emulsion with a percentage ranging from 0.1% to 20% by weight relative to the total weight of said emulsion.

The rheology agent may especially be an agent for thickening the aqueous phase, a gelling agent or a suspending agent, for example plant-derived gums such as gum arabic, konjac gum, guar gum or derivatives thereof; gums extracted from algae such as alginates; gums resulting from microbial fermentation such as xanthans, mannans, scleroglucans or derivatives thereof, cellulose and derivatives thereof such as carboxymethylcellulose or hydroxyethylcellulose; starch and derivatives thereof such as in particular modified starches, particularly acetylated, carboxymethylated, octenylsuccinates or hydroxypropylated; synthetic polymers such as polyacrylic acids or carbomers. Preferably, the rheology agent is an agent for thickening the aqueous phase. More preferentially, the rheology agent is the product Beaute by Roquette® DS112 marketed by Roquette Freres. The at least one rheology agent may be present with a percentage of 0.1% to 20%, by weight relative to the total weight of the emulsion.

Surfactants

The emulsion according to the invention may also comprise one or more surfactants, preferably selected from water-in-oil (W/O) or oil-in-water (O/W) emulsifiers. The at least one surfactant may be present with a percentage of 1% to 15%, preferentially 1% to 10%, by weight relative to the total weight of said emulsion.

The oil-in-water (O/W) emulsifier may particularly be selected from sorbitan esters which could be polyethoxylated, glycerol fatty acid esters, sucrose fatty acid esters or polyesters, polyethylene glycol fatty acid esters, polyether-modified polysiloxanes, polyethyleneglycol fatty alcohol ethers, alkyl polyglycosides and hydrogenated lecithin, this list being non-limiting. Preferably, the oil-in-water (O/W) emulsifying agent is selected from alkyl polyglycosides. More preferentially, the oil-in-water (O/W) emulsifier is the cetearyl alcohol/cetearyl glucoside marketed under the name of Montanov 68® by Seppic. The oil-in-water emulsifier may be present with a percentage of 1% to 15%, preferentially of 1% to 10%, by weight relative to the total weight of said emulsion.

The water-in-oil (W/O) emulsifier may be selected from non-ethoxylated polyol fatty esters, and particularly from non-ethoxylated fatty esters of glycerol, of polyglycerols, of sorbitol, of sorbitan, of anhydrohexitols,such as in particular isosorbide, mannitol, xylitol, erythritol, maltitol, sucrose, glucose, polydextrose, hydrogenated glucose syrups, dextrins and hydrolyzed starches. The water-in-oil emulsifier may be present with a percentage of 1% to 15%, preferentially of 1% to 10%, by weight relative to the total weight of said emulsion.

The W/O emulsifier may be selected from non-ethoxylated polyol fatty esters obtained from fatty acids or by transesterification from oil or oil mixtures. The fatty acids used comprise from 8 to 22 carbon atoms, preferably from 10 to 18 carbon atoms, and in particular from 12 to 18 carbon atoms. These acids may be linear or branched, saturated or unsaturated and have one or more lateral hydroxyl functions. The oils may be saturated or unsaturated, from liquid to solid at room temperature, and optionally have hydroxyl functions, preferably an iodine value of between 1 and 145 and in particular from 5 to 105.

The W/O emulsifier may be advantageously selected from the emulsifying systems consisting of a cyclodextrin and a water-in-oil emulsifier of natural origin, such as the emulsifying system marketed by Roquette Freres under the name Beaute by Roquette® DS146.

Dyestuff

The emulsion according to the invention may further comprise at least one dyestuff selected from water-soluble or fat-soluble dyes, fillers with the effect of coloring and/or opacifying the emulsion and/or coloring the lips, such as pigments, pearlescents, lacquers (water-soluble dyes adsorbed on an inert mineral support) and mixtures thereof. These dyestuffs may be optionally surface-treated by a hydrophobic agent such as silanes, silicones, fatty acid soaps, C9-C15 fluoroalcohol phosphates, acrylate/dimethicone copolymers, C9-C15 fluoroalcohol phosphate/silicon mixed copolymers, lecithins, carnauba wax, polyethylene, chitosan and optionally amino acids which could be acylated such as lauroyl lysine, disodium stearoyl glutamate and aluminum acyl glutamate. The pigments may be mineral or organic, natural or synthetic. Examples of pigments are especially iron, titanium or zinc oxides, and also composite pigments and goniochromatic, pearlescent, interference, photochromic or thermochromic pigments, with this list being non-limiting.

Preferably the at least one dyestuff is selected from water-soluble or fat-soluble dyes, pigments, pearlescents, lacquers and mixtures thereof. The at least one dyestuff may be present in the emulsion with a percentage of 0.1% to 30%, by weight relative to the total weight of said emulsion. The pigments and pearlescents may be present with a percentage of 0.1% to 30% by weight relative to the total weight of said emulsion. The water-soluble dyes may be present with a percentage of 0.1% to 5% by weight relative to the total weight of said emulsion.

Fillers

The emulsion according to the invention may further contain at least one filler. This term is intended to mean any particle of any form (particularly spherical or lamellar), mineral or organic, which are insoluble in the emulsion. Examples of fillers are talc, mica, silica, kaolin, boron nitride, starch, starch modified with octenylsuccinic anhydride, polyamides, silicone resins, silicone elastomer powders and acrylic polymer powders, in particular powders of poly(methyl methacrylate) or powders of styrene acrylate copolymer (Sunsphere Powders from Dow). The at least one filler may be present in the emulsion with a weight percentage of 0.1% to 30% by weight relative to the total weight of said emulsion.

Moisturizing Agent

The emulsion according to the invention may additionally comprise at least one moisturizing agent, preferentially selected from isosorbide, sorbitol, xylitol, glycerin, glycerol, propylene glycol, butylene glycol, pentylene glycol, propanediol. Preferably, the moisturizing agent is isosorbide. The at least one moisturizing agent may then be present in the emulsion with a weight percentage of 0.1% to 20% by weight relative to the total weight of said emulsion.

Other Additives

The emulsion according to the invention may contain additives that are commonly used in the field of cosmetics. As examples of additives we may mention for example preservatives, perfumes or fragrances or essential oils. Preferably, the emulsion may comprise at least one preservative and at least one perfume or fragrance or an essential oil. The percentages of these different additives are those conventionally used in the field in question. Preferably, the preservative is phanoxyethanol/Chlorphenesin/glycerin marketed under the name of Microcare PHC® by Thor. The at least one preservative is present in the emulsion with a percentage of 0.1% to 5% by weight relative to the total weight of said emulsion. The at least one perfume or fragrance or essential oil is present in the emulsion with a percentage of 0.1% to 20%, by weight relative to the total weight of said emulsion.

Evaluation of the Sensory Profile Using the “EBltouch®” Kit

The sensory profile is evaluated by a panel of trained persons using the “EBltouch®” kit sold by the Ecole de Biologie Industrielle, located at 49 avenue des Genottes, CS90009, 95895 Cergy-Pontoise, France. The panel consists of 18 persons aged 20 to 23 years.

The “EBltouch®” kit comprises reference products, referred to as “mark” for each sensory evaluation criterion, referred to as “descriptor”. For each of these descriptors, the kit includes a mark “0”, which corresponds to the minimum sensation of the descriptor, and a mark “10”, which corresponds to the maximum sensation of the descriptor.

Each panelist applies the products to be evaluated (at least two) onto the anterior face of their forearm, spreading around 0.02 grams of each product into a disc with a diameter equal to around 5 cm. All the descriptors are evaluated at the same time, during the following six steps: product appearance, holding the product, product application, immediately after product application, one minute after application, two minutes after application.

The panelists' evaluations are processed statistically to establish, for each descriptor, an average, which is the “score” of the descriptor, and a standard deviation, and the value of the parameter “p”.

In a surprising and unexpected manner, the emulsion according to the invention has, according to an evaluation by a sensory panel using the “EBltouch®” kit:
a cushion effect having a score greater than or equal to 7, preferentially greater than or equal to 7.5,
and/or a softness having a score greater than or equal to 7, preferentially greater than or equal to 7.5,
and/or a powderiness having a score greater than or equal to 5.0, preferentially greater than or equal to 5.5,
and/or a smoothness having a score greater than or equal to 5.0, preferentially greater than or equal to 5.5.

By virtue of the use according to the invention, the oil-in-water or water-in-oil emulsion, has a whipped cream texture.

Sensory Agent with “Cushion” Effect and Softness

In view of the preceding description of the use according to the invention, “sensory agent with cushion effect and softness” is understood to be a functional ingredient able to grant an emulsion, whether oil-in-water or water-in-oil, a sensory profile having a “cushion effect” descriptor and a “softness” descriptor, preferentially having scores greater than or equal to 7 according to the evaluation by a sensory panel using the “EBltouch®” kit.

### EXAMPLES

### Example 1

### Preparation of an Oil-in-Water Emulsion of “Whipped Cream” Type

An oil-in-water emulsion of “whipped cream” type according to the composition of Table 1 is prepared following the protocol below. The sensory agent with cushion effect and softness is Glycolys® from Roquette Freres. This is a carboxymethylated starch.

TABLE 1

| Phase | Trade name (supplier) | INCI name | Wt % |
|---|---|---|---|
| A1 | Demineralized water | Aqua | Q.s.p 100 |
| A1 | Polysorb ® LP | Isosorbide | 10.00 |
| | (Roquette Frères) | | |
| A2 | Beauté by Roquette ® | Acetate starch (and) | 2.00 |
| | DS112 (Roquette Frères) | Hydroxyethylcellulose (and) | |
| | | Xanthan gum | |
| B | Miglyol coco 810 | Coco-caprylate/Caprate | 5.00 |
| | (lOl oleo GmbH) | | |
| B | Miglyol T-C7 | Triheptanoin | 3.00 |
| | (lOl oleo GmbH) | | |
| B | Lexfeel N5 (lnolex) | Diheptyl Succinate (and) | 5.00 |
| | | Capryloyl Glycerin/Sebacic | |
| | | Acid Copolymer | |
| B | EmogreenTM L15 | C15-19 Alkane | 1.50 |
| | (Seppic) | | |
| B | Montanov 68 (Seppic | Cetearyl Alcohol (and) | 2.00 |
| | | Cetearyl Glucoside | |
| C | Glycolys (Roquette | Sodium Carboxymethyl | 3.00 |
| | Frères) | Starch | |
| D | Microcare PHC (Thor | Phanoxyethanol (and) | 1.00 |
| | | Chlorphenesin (and) Glycerin | |
| E | Nova Hypo Free | Fragrance | 0.10 |
| | 1114984 (Robertet) | | |

The ingredients of phase A1 are mixed and heated to 80° C. under gentle stirring with the deflocculating blade at 250-500 rpm. Phase A2 is dispersed in phase A1 under stirring with the deflocculating blade at 500-1000 rpm, maintaining a temperature of 80° C. and under stirring for 40 minutes. Separately, phase B is prepared by mixing the ingredients and heating to 80° C.

By maintaining the temperature of 80° C., phase B is emulsified in phase A1+A2 under stirring with the deflocculating blade at 2000-3000 rpm during 10 minutes. The mixture is left to cool naturally to 20-25° C. under stirring at 500-1000 rpm. Phase C is added under stirring at 500-1000 rpm at 20-25° C., and then maintained during 5-10 minutes. Optionally, phase D and E is added at the end under stirring at 500-1000 rpm.

Following the same protocol, an oil-in-water emulsion is prepared according to the composition of Table 1 without Glycolys® by Roquette. This produces a cream, with which the cream according to the invention is compared.

### Example 2

### Evaluation of the Sensory Profile by a Panel

The sensory profiles of the oil-in-water emulsion without Glycolys® and that with Glycolys®, of example 1, are evaluated by a panel of eighteen persons who are experts in the analysis of the texture of cosmetic products, using the “EBltouch®” sensory evaluation kit, sold by the Ecole de Biologie Industrielle, located at 49 avenue des Genottes, CS90009, 95895 Cergy-Pontoise, France.

Appearance Descriptors:

The three first descriptors evaluated are the visual characteristics of the product. The product is examined in the jar, under a lamp provided with a bulb at a temperature of 5500 K.

The “opaque” descriptor of a product is defined by whether or not the product lets the light pass through it.

The “whiteness” descriptor is defined by the reference color palette of the “EBltouch®” kit. The product is compared with the color palette by a panel of evaluators.

The “shiny” descriptor is defined by whether or not the product reflects light.

Holding Descriptors:

The following descriptors are evaluated when the product is picked up. The examination of 50 to 100 μL of the studied product is carried out between the thumb and the index finger, under the lamp.

The “fluidity” descriptor is evaluated when pressure is applied to the product, between the thumb and the index. If there is no resistance, and it flows off the fingers and moves away from the pressure zone, it is fluid; if the opposite is true, it is non-fluid.

The “cushion effect” descriptor is evaluated by applying two gentle pressures on the product between the thumb and the index finger. When a bounce or a dampening or a cushion is felt under the fingers, this is called a cushion effect. The amplitude of the bounce or dampening is evaluated by a score out of 10:0 corresponds to the “fluid mark” reference of the EBltouch® kit, 10 corresponds to the “repairing hand cream for damaged hands, with allantoin” by “Mixa”.

The “threading” descriptor is evaluated by placing the product between the thumb and the index finger and slowly separating the two fingers, allowing a continuous thread to form between the two fingers. The thread breaks when the fingers move too far apart.

The “slippery” descriptor is evaluated by placing the product between the thumb and the index finger so that there is no resistance when performing a rubbing movement (like “snapping the fingers”). The product facilitates movement of the two fingers.

Application Descriptors:

When the product is applied by spreading, three other descriptors are evaluated. The product is examined, after having deposited 50 to 100 μl of the studied product on the hand, under the lamp, during the spreading thereof in 10 rotations.

The “softness” descriptor is evaluated during the application, from the 2nd to the 5th rotation. When there is a sensation of depression or thickness, this is referred to as softness. The amplitude of the softness is evaluated by a score out of 10:0 corresponds to the “Fluid” mark of the EBltouch® kit, 10 corresponds to the “Non-opaque” mark of the EBltouch® kit.

The “whitening” descriptor is evaluated during the application, from the 2nd to the 5th rotation: the product whitens the skin.

The “spread” descriptor is evaluated between the 5th and 10th rotation: there is no resistance to movement.

The final descriptors are evaluated after having carried out the 10 rotations.

Descriptors Immediately after Application:

The “powderiness” descriptor is evaluated between the 2nd and 5th rotations. The fingers slide well over the skin. The product is perceived as a powdery substance on the skin. The amplitude of the powderiness is evaluated by a score out of 10:0 corresponds to the “Slippery” mark of the EBltouch® kit, 10 corresponds to “The POREfessionnal: Pearl Primer” foundation by “Benefit”

Descriptors after 1 Minute:

30

For the four following descriptors, the examination is carried out under the lamp, on the skin, 1 minute after spreading 50 to 100 μl of the product.

The “smooth” descriptor is evaluated by sliding over the skin: a dry and slippery sensation is felt.

The “sticky” descriptor is evaluated by three movements pressing on the skin with the index finger: adhesion is felt.

The “greasy” descriptor is evaluated by pinching the skin between the thumb and the index finger: the thickness of the product between the fingers is evaluated.

The improved criteria for sensory properties in the emulsion according to the invention are: the cushion effect, the softness, the powderiness, the smoothness.

Table 2 shows the sensory profiles evaluated for an oil-in-water emulsion without starch according to the invention, and for an oil-in-water emulsion with a starch according to the invention (Glycolys®), as prepared in example 1.

TABLE 2

| Sensory | Emulsion | Emulsion with |
|---|---|---|
| criterion | without Glycolys | Glycolys |
| Opaque | 10.00 | 10.00 |
| White | 9.94 | 10.00 |
| Shiny | 8.94 | 7.81 |
| Fluidity | 8.88 | 1.44 |
| Cushion effect | 1.06 | 7.94 |
| Threading | 1.38 | 3.00 |
| Slippery | 9.25 | 7.31 |
| Softness | 3.25 | 7.63 |
| Whitening | 1.06 | 3.31 |
| Spread | 8.56 | 5.75 |
| Powderiness | 0.69 | 5.63 |
| Softness | 3.50 | 6.00 |
| Sticky | 3.75 | 2.94 |
| Greasy | 6.25 | 7.00 |
| No residue | 6.94 | 7.06 |

The sensory panel made it possible to demonstrate that the Glycolys® cross-linked carboxyalkylated starch makes it possible to obtain an oil-in-water emulsion having a cushion effect, a softness, a powderiness and a smooth touch.

1. A non-therapeutic cosmetic use of a carboxyalkylated granular starch in an oil-in-water or a water-in-oil emulsion, preferentially oil-in-water, as a sensory agent having a cushion effect and a softness effect.

2. The non-therapeutic cosmetic use according to claim 1, wherein the sensory profile of said cosmetic preparation has a cushion effect, and/or a softness.

3. The non-therapeutic cosmetic use according to claim 1, wherein the carboxyalkylated granular starch is selected from the carboxymethylated starches, the carboxyethylated starches, the carboxypropylated starches.

4. The non-therapeutic cosmetic use according to claim 1, wherein the carboxyalkylated granular starch is also cross-linked by a cross-linking agent.

5. The non-therapeutic cosmetic use according to claim 1, wherein the cross-linking agent is selected from the polyfunctional acids, the polyacid anhydrides, or the polyfunctional basic organic molecules, as well as the metallic salts thereof.

6. The non-therapeutic cosmetic use according to claim 1, wherein the starch is a starch from corn, potato, wheat, rice, peas, oats, lentils, faba beans, broad beans, beans, chickpeas, cassava, or combinations thereof.

7. The non-therapeutic cosmetic use according to claim 1, wherein the oil-in-water or water-in-oil emulsion has a whipped cream texture.

8. An oil-in-water (O/W) or water-in-oil (W/O) emulsion, comprising:
a fatty phase comprising 10 to 70% by weight of at least one non-volatile oil selected from the fatty esters, the glycol esters, the higher fatty acids, the higher fatty alcohols, the vegetable butters, the hydrocarbon or silicon oils,
an aqueous phase comprising 30 to 90% by weight of water; and 1% to 20%, preferably 3 to 18%, most preferentially 5% to 15% by weight of carboxyalkylated granular starch selected from the carboxymethylated starches, the carboxyethylated starches, and the carboxypropylated starches,
1% to 15%, preferentially 1% to 10%, by weight of at least one surfactant, the percentages being expressed by weight relative to the total weight of said emulsion.

9. The emulsion according to claim 8, wherein the fatty phase further comprises volatile oils selected from silicone oils or short fatty acid triglycerides.

10. The emulsion according to claim 8, wherein the fatty phase further comprises at least one fatty phase structuring agent such as a lipophilic gelling agent, a wax and/or a paste-like compound.

11. The emulsion according to claim 8, wherein the aqueous phase further comprises one or more water-miscible organic solvents, and/or one or more rheology agents.

12. The emulsion according to claim 8, wherein the at least one surfactant is an oil-in-water surfactant selected from optionally polyethoxylated sorbitan esters, glycerol and fatty acid esters, sucrose and fatty acid esters or polyesters, polyethylene glycol and fatty acid esters, polyether-modified polysiloxanes, polyethylene glycol and fatty alcohol ethers, alkyl polyglycosides and hydrogenated lecithin.

13. The emulsion according to claim 8, further comprising at least one dyestuff selected from water-soluble or fat-soluble dyes, pigments, pearlescents, lacquers, and mixtures thereof.

14. The emulsion according to claim 8, wherein the carboxyalkylated granular starch selected from carboxymethylated starches, carboxyethylated starches, and carboxypropylated starches is also cross-linked by a cross-linking agent.

15. The emulsion according to claim 8, wherein the starch is a starch from corn, potato, wheat, rice, peas, oats, lentils, faba beans, broad beans, beans, chickpeas, cassava, or combinations thereof, preferentially is a potato starch.
